(19) United States
(12) Patent Application Publication
(10) Pub. No.: US 2019/0374520 A1
(43) Pub. Date: Dec. 12, 2019

(54) TREATMENT OF MIGRAINE

(71) Applicant: Allergan Pharmaceuticals International Limited, Dublin 17 (IE)

(72) Inventors: Joel Trugman, Hoboken, NJ (US); Michelle Finnegan, Brookyln, NY (US)

(21) Appl. No.: 16/433,298

(22) Filed: Jun. 6, 2019

Related U.S. Application Data
(60) Provisional application No. 62/682,656, filed on Jun. 8, 2018.

(51) Int. Cl. A61K 31/4375; A61P 25/06; A61K 9/00

(52) U.S. Cl. CPC A61K 31/4375; A61K 9/0053; A61P 25/06

(57) ABSTRACT

The application provides prophylactic methods for the treatment of migraine by the administration of a CGRP antagonist, preferably atogepant or ubrogepant, or a pharmaceutically acceptable salt thereof.

### FIELD

The application is related to medicaments and methods for treating migraine.

### BACKGROUND

Migraine is a highly prevalent, severe, and disabling neurological condition with a significant unmet need for effective treatments. (Holland, P. R. & Goadsby, P. J. Neurotherapeutics (2018). Migraine represents a significant burden to patients and society.

CGRP (Calcitonin Gene-Related Peptide) is a naturally occurring 37-amino acid peptide that is generated by tissue-specific alternate processing of calcitonin messenger RNA and is widely distributed in the central and peripheral nervous system. Calcitonin gene-related peptide (CGRP) is a potent vasodilatory neurotransmitter believed to play a key role in migraine pathophysiology. The initial human clinical validation of the CGRP target was provided by Boehringer Ingelheim in 2003 with the report that an IV formulation comprising olcegepant was efficacious in the acute treatment of migraine and the mechanism was confirmed by a study using telcagepant (a CGRP antagonist) in an oral formulation. The first clinically tested CGRP antagonists, olcegepant, was based on a dipeptide backbone, had high molecular weight, and was not orally bioavailable. Following the success of olcegepant, a number of orally acting CGRP antagonists were advanced to clinical trials including MK-3207 and telcagepant. Some of the CGRP antagonists resulted in elevated levels of liver transaminases in some patients. The elevated liver enzyme levels were observed for MK-3207 and telcagepant. It would be advantageous to develop CGRP antagonists that do not elevate liver enzyme levels significantly.

### SUMMARY

In some embodiments, the application provides prophylactic methods for the treatment of migraine by the administration of a prophylactically effective amount of atogepant or ubrogepnt or a pharmaceutically acceptable salt thereof. In some embodiments, the prophylactic treatment with atogepant or ubrogepant does not significantly affect the level of liver enzymes. Some embodiments provide methods for treating or reducing migraine comprising the step of administering to a patient in need thereof, an effective amount of ubrogepant or atogepant, or a pharmaceutically acceptable salt, ester or prodrug thereof, wherein the treatment provides significant reduction in the number of migraine days per month, without significantly affecting the liver enzyme levels.

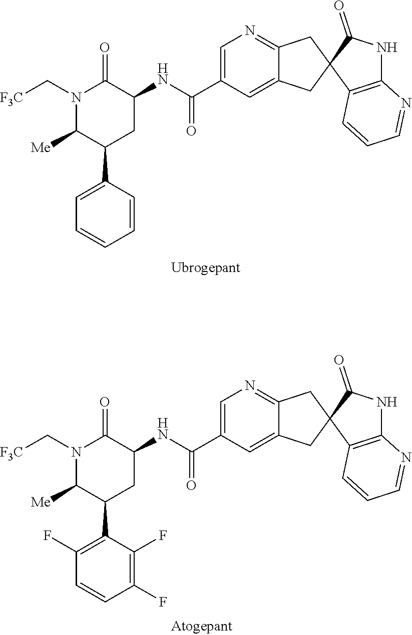 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F ; C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

### BRIEF DESCRIPTION OF THE FIGURES

FIG. 1 shows the percentage of patients reporting at least 75% reduction in migraine or probably migraine days after one month, two months or three months of treatment with placebo or 10 (QD), 30 (QD), 60 (QD), 30 (BID) or 60 (BID) doses of atogepant.

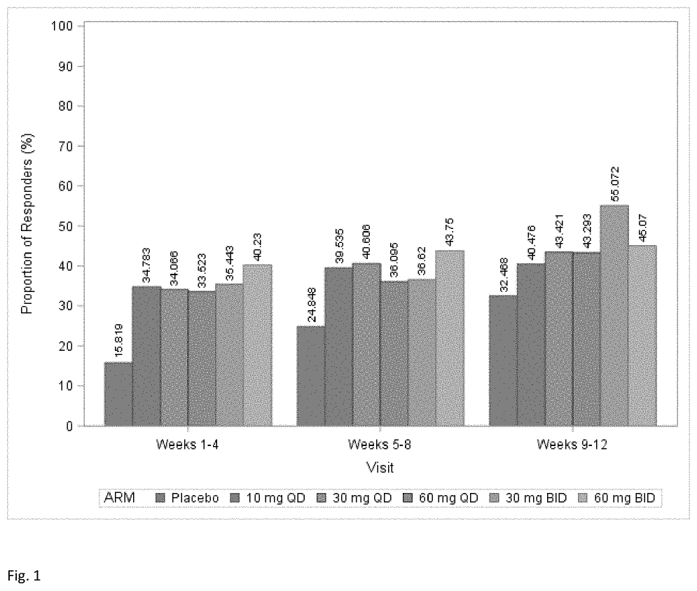

FIG. 2 shows the percentage of patients reporting at 100% reduction in migraine or probably migraine after one month, two months or three months of treatment with placebo or 10 (QD), 30 (QD), 60 (QD), 30 (BID) or 60 (BID) doses of atogepant.

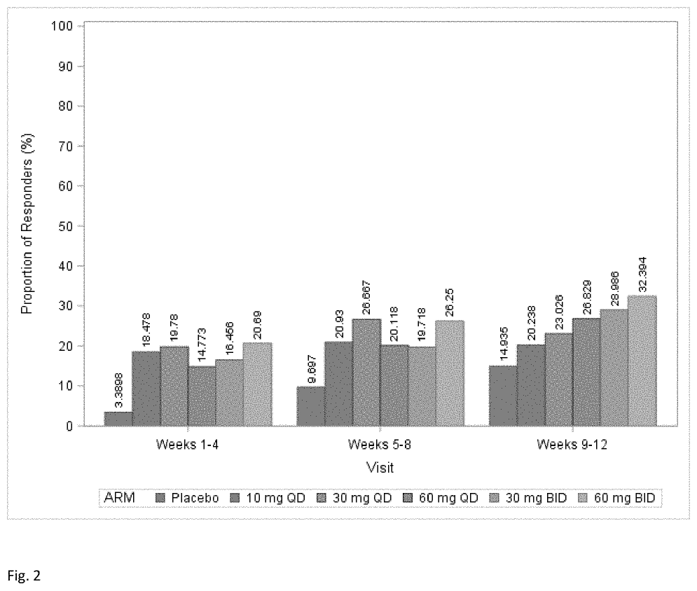

### DETAILED DESCRIPTION

The application provides methods for the treatment of migraine for a patient in need thereof. The application provides prophylactic treatment of patients suffering from migraine by the administration of a prophylactically effective amount atogepant or ubrogepant or a pharmaceutically acceptable salt thereof;

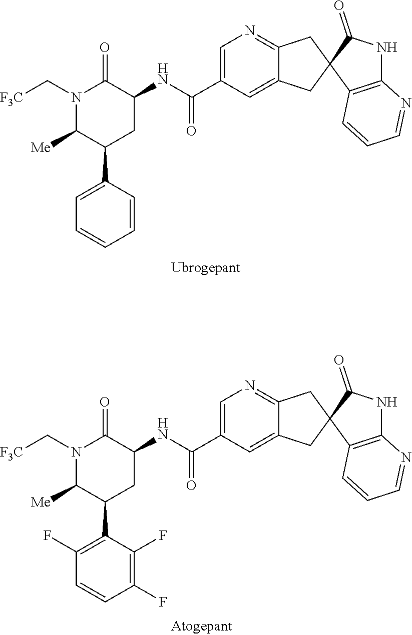 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F ; C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

Prophylactic treatment can reduce the frequency and intensity of migraine attacks. In some embodiments, the prophylactic methods can result in complete freedom from symptoms associated with migraine attacks, including headaches. In some embodiment, the administration of atogepant or ubrogepant may provide for fewer symptoms or symptoms of reduced intensity. In some embodiments the non-headache symptoms of migraine that are reduced or eliminated. The prophylactic methods of the present invention are directed to the entire range of symptoms experienced by a patient during a migraine attack, and not merely at the prevention of headaches associated with a migraine attack.

The indications for preventative treatment of migraine have been published by the American Academy of Neurology. (See Update: Pharmacologic Treatment for Episodic Migraine Prevention in Adults, American Academy of Neurology, 2012.) Prophylactic treatment is generally proposed for patients who suffer from two or more migraine attacks per month. Prophylactic treatment can also be used for patients who experience less frequent migraine attacks that are more potent or even disabling.

In some embodiments, the patient suffers from one or more symptoms of migraine selected from sinusitis, nausea, nasopharangytis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light, sounds, or smells, worsening pain during physical activity, and vomiting, abdominal pain or heartburn, loss of appetite, lightheadedness, blurred vision, and fainting. In some embodiments, the administration of a prophylactically effective amount of atogepant or ubrogepant results in the improvement of reduced frequency or intensity of symptoms.

In one embodiment, the present invention provides a method for prophylactic treatment for migraine comprising regularly administering to a patient in need thereof a therapeutically effective amount of atogepant or ubrogepant, or a pharmaceutically acceptable salt thereof.

In some embodiments the CGRP antagonist is atogepant. In some embodiments, atogepant is administered at an oral dose of about 5 to about 500 mg once, twice or three times a day. In some embodiments, atogepant is administered orally at a once daily dose of about 10 mg. In some embodiments, atogepant is administered orally at a once daily dose of about 30 mg. In some embodiments, atogepant is administered orally at a once daily dose of about 50 mg. In some embodiments, atogepant is administered orally at a once daily dose of about 100 mg. In some embodiments, atogepant is administered orally at a dose of about 10 mg twice a day. In some embodiments, atogepant is administered orally at a dose of about 30 mg twice a day. In some embodiments, atogepant is administered orally at a dose of about 50 mg twice a day. In some embodiments, atogepant is administered orally at a dose of about 100 mg twice a day.

In some embodiments, the CGRP antagonist is ubrogepant. In some embodiments, ubrogepant is administered at an oral dose of about 5 to about 500 mg once, twice or three times a day. In some embodiments, ubrogepant is administered at an oral dose of about 25 mg once, twice or three times a day. In some embodiments, ubrogepant is administered at an oral dose of about 50 mg once, twice or three times a day. In some embodiments, ubrogepant is administered at an oral dose of about 100 mg once, twice or three times a day. In some embodiments, ubrogepant is administered at an oral dose of about 200 mg once, twice or three times a day.

In one embodiment, ubrogepant is administered at a dose of about 1-1000 mg per day. In one embodiment, ubrogepant is administered at a dose of about 5, 10, 25, 50, 100, 200 or 400 mg per day.

In some embodiments, the CGRP-antagonist can be administered orally, sublingually, transdermally, subcutaneously, intravenously, or intramuscularly.

In some embodiments, the prophylactic treatment with atogepant or ubrogepant does not significantly affect the level of liver enzymes, in particular alanine aminotransferase (ALT) or aspartate aminotransferase (AST). For example, after treatment with atogepant or ubrogepant for a period of about three months, the baseline pretreatment level of AST or ALT is not increased more than 50%, 75%, 100% or 200%. The baseline ALT levels can be measured as units per liter in the serum based on established methods. (Neuschwander-Tetri, B. A. et. al., Arch Intern Med. 2004 Mar. 24; 168(6): 663-666; Kasarala, G. et. al., Clinical Liver Disease, 8, (1) July 2016) Neuschwander-Tetri states that the healthy ALT should be up to 30 units per liter of serum (U/L) for men, and 19 U/L for women. In some embodiments, the prophylactic treatment with atogepant or ubrogepant for a period of about three months does not elevate the ALT levels above the healthy levels of 30 U/L for men and 19 U/L for women. In one embodiment, the application provides a method of treating migraine in patients with elevated ALT or AST levels. For example, migraine in patients with elevated ALT or AST levels (above 30 U/L for men and 19 U/L for women) can be treated with atogepant or ubrogepant. In some embodiments, the application provides method for treating migraine in patients with nonalcoholic steatohepatitis (NASH).

In some embodiments, the pretreatment baseline level of AST is about 5 to 40 units per liter of serum, and after treatment with atogepant or ubrogepant for a period of about three months, the level of AST is less than about 100 or 90 or 75 or 60 or 50 units per liter of serum.

In some embodiments, the pretreatment baseline level of ALT and pretreatment baseline level of AST is about 7 to 56 units per liter of serum, and after treatment with atogepant or ubrogepant for a period of about three months, the level of ALT is less than about 100 or 90 or 75 or 60 or 50 units per liter of serum.

In some embodiments, ubrogepant or atogepant, or a pharmaceutically acceptable salt, ester or prodrug thereof, is administered to a patient identified as susceptible to treatment with ubrogepant or atogepant or pharmaceutically acceptable salt, ester or prodrug thereof. A patient that suffers from episodic migraine can be considered susceptible to treatment with ubrogepant or atogepant, or pharmaceutically acceptable salt, ester or prodrug thereof, if after treatment with atogepant (for example 10 mg (QD or BID), 30 mg (QD or BID) or 60 mg (QD or BID)), or ubrogepant (for example 25 mg (QD or BID), 50 mg (QD or BID) or 1000 mg (QD or BID)) for a period of one month, two months or three months, the patient achieves 70%, preferably 75%, more preferably 80%, more preferably 90%, or more preferably 100% reduction in migraine or probable migraine days.

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“CGRP”, abbreviated for Calcitonin-Gene-Related-Peptide, as used herein encompasses any member of the calcitonin family, including any calcitonin gene related peptide and analogs, calcitonin, amylin, adrenomedullin and their analogs.

“CGRP antagonist” refers to any molecule that exhibits any one or more of the following characteristics: (a) bind to CGRP or CGRP-R and the binding results in a reduction or inhibition of CGRP activity; (b) block CGRP from binding to its receptor(s); (c) block or decrease CGRP receptor activation; (d) inhibit CGRP biological activity or downstream pathways mediated by CGRP signaling function; (e) increase clearance of CGRP; and (f) inhibit or reduce CGRP synthesis, production or release. CGRP antagonists include but are not limited to antibodies to CGRP, antibodies to the CGRP-R, small molecules that antagonize CGRP, and small molecules that antagonize CGRP-R.

The term “prophylactic” refers to the prevention of the onset, recurrence or spread of a disease or disorder, or of one or more symptoms associated with the disease or disorder. In one embodiment, such symptoms are those known to a person of skill in the art to be associated with the disease or disorder being prevented. In certain embodiments, the terms refer to the treatment with or administration of a compound provided herein, with or without other additional active compound, prior to the onset of symptoms, particularly to patients at risk of disease or disorders provided herein. The terms encompass the inhibition or reduction of a symptom of the particular disease. For frequent migraines, prophylactic treatments are employed to reduce the frequency of migraines and also to reduce the severity and duration of migraines and their associated symptoms when they occur.

A “prophylactically effective amount” of a compound is an amount sufficient to prevent a disease or disorder, or prevent its recurrence. A prophylactically effective amount of a compound means an amount of therapeutic agent that can provide a prophylactic benefit in the prevention of the disease. The term “prophylactically effective amount” can encompass an amount that improves overall prophylaxis or enhances the prophylactic efficacy of another prophylactic agent.

“Effective amount” as applied to the biologically active ingredient means that amount of the ingredient which is generally sufficient to effect a desired change in the subject. For example, where the desired effect is a reduction in an autoimmune disorder symptom, an effective amount of the ingredient is that amount which causes at least a substantial reduction of the autoimmune disorder symptom, and without resulting in significant toxicity.

“Intramuscular” or “intramuscularly” means into or within (as in administration or injection of a CGRP antagonist into) a muscle.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

“Patient” means a human or non-human subject receiving medical or veterinary care. Accordingly, the compositions as disclosed herein can be used in treating any animal, such as, for example, mammals, or the like.

“Peripherally administering” or “peripheral administration” means subdermal, intradermal, transdermal, or subcutaneous administration, but excludes intramuscular administration. “Peripheral” means in a subdermal location, and excludes visceral sites.

“Pharmaceutical composition” means a composition comprising an active pharmaceutical ingredient, such as, for example, a CGRP antagonist, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two-component system, for example a vacuum-dried composition reconstituted with a reconstitution vehicle which can, for example, contain an ingredient not present in the initial compounding of the pharmaceutical composition. A two-component system can provide several benefits, including that of allowing incorporation of ingredients which are not sufficiently compatible for long-term shelf storage with the first component of the two-component system. A pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

“Tonicity agent” means a low molecular weight excipient which is included in a formulation to provide isotonicity. Disaccharide, such as trehalose or sucrose, polyalcohol, such as sorbitol or mannitol, monosaccharide, such as glucose, and salt, such as sodium chloride, can serve as a tonicity agent.

“Polysaccharide” means a polymer of more than two saccharide molecule monomers. The monomers can be identical or different.

“Stabilizers” can include excipients, and can include protein and non-protein molecules.

“Therapeutic formulation” means a formulation can be used to treat and thereby alleviate a disorder or a disease, such as, for example, a disorder or a disease characterized by hyperactivity (i.e. spasticity) of a peripheral muscle.

“Treating” means to alleviate (or to eliminate) at least one symptom of a condition or disorder, such as, for example, sinusitis, nausea, nasopharangytis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light, sounds, or smells, worsening pain during physical activity, and vomiting, abdominal pain or heartburn, loss of appetite, lightheadedness, blurred vision or fainting or the like, either temporarily or permanently.

### EXAMPLES

The following non-limiting examples provide those of ordinary skill in the art with possible case scenarios and specific methods to treat conditions within the scope of the present disclosure and are not intended to limit the scope of the disclosure.

In a multicenter, randomized, double-blind, placebo-controlled, parallel-group study designed to evaluate the efficacy, safety, and tolerability of multiple doses and doses regimens of atogepant for the prevention of migraine. The patients were randomized (2:1:2:1:2:1) to 1 of 6 treatment groups: placebo, atogepant 10 mg QD, atogepant 30 mg QD, atogepant 30 mg BID, atogepant 60 mg QD, and atogepant 60 mg BID.

This study consisted of a 4-week screening and baseline period and a 12-week double-blind treatment period, which was followed by a 4-week safety follow-up period. Total study duration is 20-weeks.

To be randomized, eligible patients had to be 18 to 75 years of age (inclusive), have a history of migraine with or without aura for at least 1 year consistent with a diagnosis according to the International Classification of Headache Disorders criteria, 3rd edition, beta version, have experienced 4 to 14 migraine/probable migraine headache days and <15 headache days in the 28-day baseline period. Patients who had clinically significant hematologic, endocrine, cardiovascular, cerebrovascular, pulmonary, renal, hepatic, gastrointestinal, or neurologic disease were excluded from the study.

A total of 834 patients were randomized to double-blind treatment (ITT population), and 825 patients took at least 1 dose of double-blind IP (safety population). A total of 795 treated patients had an evaluable baseline period of diary data and at least 1 evaluable post-baseline 4-week interval (Weeks 1-4, 5-8, or 9-12) of diary data. The primary efficacy variable was the change from baseline in mean monthly migraine/probable migraine headache days across the 12-week treatment period. The secondary efficacy variables were the change from baseline in mean monthly headache days across the 12-week treatment period, proportion of patients with at least a 50% reduction in mean monthly migraine/probable migraine headache days across the 12-week treatment period, and the change from baseline in mean monthly acute medication use days across the 12-week treatment period.

Results from the study are presented in tables below:

Error! No text of specified style in document.

TABLE
Primary Endpoint: Change from
Baseline in Mean Monthly MPM Headache Days across the 12-Week
Treatment Period - mITT Population

| | | Atogepant | | | Atogepant | Atogepant |
|---|---|---|---|---|---|---|
| | | 10 mg | Atogepant | Atogepant | 30 mg | 60 mg |
| | Placebo | QD | 30 mg QD | 60 mg QD | BID | BID |
| Statistics | (N = 178) | (N = 92) | (N = 182) | (N = 177) | (N = 79) | (N = 87) |
| Baseline | | | | | | |
| Mean | 7.81 | 7.63 | 7.64 | 7.74 | 7.38 | 7.62 |
| SD | 2.51 | 2.51 | 2.37 | 2.59 | 2.43 | 2.56 |
| CFB | | | | | | |
| MMRM | | | | | | |
| LS Mean (SE) | −2.85 | −4.00 | −3.76 | −3.55 | −4.23 | −4.14 |
| | (0.23) | (0.32) | (0.23) | (0.23) | (0.35) | (0.33) |
| 95% CI | −3.30, −2.39 | −4.63, −3.36 | −4.21, −3.31 | −4.01, −3.10 | −4.92, −3.55 | −4.79, −3.48 |
| CGP vs. | | | | | | |
| Placebo | | | | | | |
| LSMD (SE) | | −1.15 | −0.91 | −0.70 | −1.39 | −1.29 |
| | | (0.40) | (0.33) | (0.33) | (0.42) | (0.41) |
| 95% CI | | −1.93, −0.37 | −1.55, −0.27 | −1.35, −0.06 | −2.21, −0.56 | −2.09, −0.49 |
| p-value | | 0.0039 | 0.0056 | 0.0325 | 0.0010 | 0.0016 |
| Adjusted p- | | 0.0236 | 0.0390 | 0.0390 | 0.0034 | 0.0031 |
| value | | | | | | |

MMRM = mixed-effects model for repeated measures for change from baseline. The model includes treatment group and visit as fixed effect, the baseline value as a covariate, and treatment group by visit and baseline by visit as interaction terms. P-values are from the test between the atogepant dose group and placebo.
Adjusted p-value: using graphic approach to control the overall type I error rate for multiple comparisons.
CFB = change from baseline,
SD = Standard Deviation,
LS = Least Squares,
SE = standard error of the least squares,
CI = Confidence Interval,
LSMD = least squares mean difference.

TABLE 2
Change from Baseline in Mean Monthly Headache Days across the 12-
Week Treatment Period - mITT Population

| | | | | Atogepant | Atogepant | Atogepant |
|---|---|---|---|---|---|---|
| | | Atogepant | Atogepant | 60 mg | 30 mg | 60 mg |
| | Placebo | 10 mg QD | 30 mg QD | QD | BID | BID |
| Statistics | (N = 178) | (N = 92) | (N = 182) | (N = 177) | (N = 79) | (N = 87) |
| Baseline | | | | | | |
| Mean | 9.07 | 8.89 | 8.74 | 8.86 | 8.71 | 8.80 |
| SD | 2.70 | 2.70 | 2.51 | 2.76 | 2.73 | 3.12 |
| CFB | | | | | | |
| MMRM | | | | | | |
| LS Mean (SE) | −2.93 | −4.31 | −4.17 | −3.86 | −4.23 | −4.32 |
| | (0.25) | (0.35) | (0.25) | (0.25) | (0.38) | (0.36) |
| 95% CI | −3.42, −2.43 | −4.99, −3.62 | −4.66, −3.68 | −4.36, −3.37 | −4.97, −3.48 | −5.03, −3.61 |
| CGP vs. | | | | | | |
| Placebo | | | | | | |
| LSMD (SE) | | −1.38 | −1.24 | −0.94 | −1.30 | −1.39 |
| | | (0.43) | (0.36) | (0.36) | (0.46) | (0.44) |
| 95% CI | | −2.23, −0.54 | −1.94, −0.55 | −1.64, −0.24 | −2.20, −0.41 | −2.26, −0.53 |
| p-value | | 0.0014 | 0.0005 | 0.0087 | 0.0044 | 0.0017 |
| Adjusted p- | | 0.0236 | 0.0390 | 0.0390 | 0.0131 | 0.0083 |
| value | | | | | | |

MMRM = mixed-effects mode for repeated measures for change from baseline. The model includes treatment group and visit as fixed effect, the baseline value as a covariate, and treatment group by visit and baseline by visit as interaction terms. P-values are from the test between the atogepant dose group and placebo.
Adjusted p-value: using graphic approach to control the overall type I error rate for multiple comparisons.
CFB = change from baseline,
SD = Standard Deviation,
LS = Least Squares,
SE = standard error of the least squares,
CI = Confidence Interval,
LSMD = least squares mean difference.

TABLE 3
Proportion of Participants with at Least 50% Reduction in Mean
Monthly MPM Headache Days across the 12-Week Treatment Period -
mITT Population

| | | | Atogepant | Atogepant | Atogepant | Atogepant |
|---|---|---|---|---|---|---|
| | | Atogepant | 30 mg | 60 mg | 30 mg | 60 mg |
| | Placebo | 10 mg QD | QD | QD | BID | BID |
| Statistics | (N = 178) | (N = 92) | (N = 182) | (N = 177) | (N = 79) | (N = 87) |
| 50% Responders | | | | | | |
| Responders, n (%) | 72 (40.4) | 53 (57.6) | 97 (53.3) | 92 (52.0) | 46 (58.2) | 54 (62.1) |
| Non-responders, n | 106 (59.6) | 39 (42.4) | 85 (46.7) | 85 (48.0) | 33 (41.8) | 33 (37.9) |
| (%) | | | | | | |
| Odds ratio vs. | | 1.50 | 1.46 | 1.42 | 1.83 | 2.03 |
| Placebo | | | | | | |
| 95% CI | | (0.98, | (1.02, | (1.00, | (1.15, | (1.30, |
| | | 2.31) | 2.08) | 2.03) | 2.91) | 3.18) |
| p-value | | 0.0617 | 0.0369 | 0.0512 | 0.0113 | 0.0019 |
| Adjusted p-value | | 0.1107 | 0.1107 | 0.1537 | 0.0339 | 0.0097 |

Non-responders include participants not met responder criteria.
n = Number of participants within a specific category.
Analyses are based on generalized linear mixed model for repeated measures. The model includes fixed factors (treatment group, analysis visit), covariates (baseline value), and interactions (treatment group by analysis visit, baseline value by analysis visit), with an unstructured covariance matrix (compound symmetry covariance matrix if convergence fails).

TABLE 4
Change from Baseline in Mean Monthly Acute Medication Use Days
across the 12-Week Treatment Period - mITT Population

| | | Atogepant | | | Atogepant | |
|---|---|---|---|---|---|---|
| | | 10 mg | Atogepant | Atogepant | 30 mg | Atogepant |
| | Placebo | QD | 30 mg QD | 60 mg QD | BID | 60 mg BID |
| Statistics | (N = 178) | (N = 92) | (N = 182) | (N = 177) | (N = 79) | (N = 87) |
| Baseline | | | | | | |
| Mean | 6.57 | 6.16 | 6.62 | 6.79 | 6.20 | 6.37 |
| SD | 3.21 | 3.31 | 3.04 | 3.27 | 3.26 | 3.41 |
| CFB | | | | | | |
| MMRM | | | | | | |
| LS Mean (SE) | −2.42 | −3.71 | −3.86 | −3.53 | −3.77 | −3.64 |
| | (0.21) | (0.29) | (0.20) | (0.21) | (0.31) | (0.29) |
| 95% CI | −2.82, −2.01 | −4.27, −3.15 | −4.26, −3.46 | −3.93, −3.13 | −4.38, −3.16 | −4.22, −3.06 |
| CGP vs. | | | | | | |
| Placebo | | | | | | |
| LSMD (SE) | | −1.30 | −1.44 | −1.11 | −1.35 | −1.22 |
| | | (0.35) | (0.29) | (0.29) | (0.37) | (0.36) |
| 95% CI | | −1.99, −0.60 | −2.01, −0.87 | −1.68, −0.54 | −2.08, −0.62 | −1.93, −0.52 |
| p-value | | 0.0002 | <0.0001 | 0.0001 | 0.0003 | 0.0007 |
| Adjusted p- | | 0.1107 | 0.1107 | 0.1537 | 0.0339 | 0.0097 |
| value | | | | | | |

MMRM = mixed-effects model for repeated measures for change from baseline. The model includes treatment group and visit as fixed effect, the baseline value as a covariate, and treatment group by visit and baseline by visit as interaction terms. P-values are from the test between the atogepant dose group and placebo.
Adjusted p-value: using graphic approach to control the overall type I error rate for multiple comparisons.
CFB = change from baseline,
SD = Standard Deviation,
LS = Least Squares,
SE = standard error of the least squares,
CI = Confidence Interval,
LSMD = least squares mean difference.

TABLE 5
Number of Participants with Postbaseline Hepatic Laboratory
Parameter Values of Clinical Interest - Safety Population

| | | | | | Atogepant | Atogepant |
|---|---|---|---|---|---|---|
| | | Atogepant | Atogepant | Atogepant | 30 mg | 60 mg |
| | Placebo | 10 mg QD | 30 mg QD | 60 mg QD | BID | BID |
| Parameter (unit) | (N = 186) | (N = 93) | (N = 183) | (N = 186) | (N = 86) | (N = 91) |
| Criterion | n/N1 (%) | n/N1 (%) | n/N1 (%) | n/N1 (%) | n/N1 (%) | n/N1 (%) |
| ALT or AST | | | | | | |
| (U/L) | | | | | | |
| >=1 * ULN | 28/179 | 17/92 (18.5) | 32/180 (17.8) | 25/181 (13.8) | 11/84 (13.1) | 9/88 (10.2) |
| | (15.6) | | | | | |
| >=1.5 * ULN | 8/179 | 4/92 (4.3) | 13/180 (7.2) | 8/181 (4.4) | 2/84 (2.4) | 1/88 (1.1) |
| | (4.5) | | | | | |
| >=2 * ULN | 5/179 | 4/92 (4.3) | 6/180 (3.3) | 5/181 (2.8) | 2/84 (2.4) | 1/88 (1.1) |
| | (2.8) | | | | | |
| >=3 * ULN | 3/179 | 2/92 (2.2) | 1/180 (0.6) | 3/181 (1.7) | 1/84 (1.2) | 1/88 (1.1) |
| | (1.7) | | | | | |
| >=5 * ULN | 3/179 | 0 | 0 | 1/181 (0.6) | 0 | 0 |
| | (1.7) | | | | | |
| >=10 * ULN | 0 | 0 | 0 | 0 | 0 | 0 |
| >=20 * ULN | 0 | 0 | 0 | 0 | 0 | 0 |
| Bilirubin Total | | | | | | |
| (umol/L) | | | | | | |
| >=1 * ULN | 4/179 | 3/92 (3.3) | 15/179 (8.4) | 8/181 (4.4) | 3/84 (3.6) | 1/88 (1.1) |
| | (2.2) | | | | | |
| >=1.5 * ULN | 1/179 | 1/92 (1.1) | 2/179 (1.1) | 2/181 (1.1) | 1/84 (1.2) | 0 |
| | (0.6) | | | | | |
| >=2 * ULN | 0 | 0 | 0 | 0 | 0 | 0 |
| >=3 * ULN | 0 | 0 | 0 | 0 | 0 | 0 |
| >=5 * ULN | 0 | 0 | 0 | 0 | 0 | 0 |
| >=10 * ULN | 0 | 0 | 0 | 0 | 0 | 0 |
| >=20 * ULN | 0 | 0 | 0 | 0 | 0 | 0 |
| Alkaline | | | | | | |
| Phosphatase | | | | | | |
| (U/L) | | | | | | |
| >=1 * ULN | 12/179 | 5/92 (5.4) | 19/179 (10.6) | 12/181 (6.6) | 2/84 (2.4) | 3/88 (3.4) |
| | (6.7) | | | | | |
| >=1.5 * ULN | 1/179 | 1/92 (1.1) | 1/179 (0.6) | 0 | 0 | 0 |
| | (0.6) | | | | | |
| >=2 * ULN | 0 | 1/92 (1.1) | 0 | 0 | 0 | 0 |
| >=3 * ULN | 0 | 0 | 0 | 0 | 0 | 0 |
| >=5 * ULN | 0 | 0 | 0 | 0 | 0 | 0 |
| >=10 * ULN | 0 | 0 | 0 | 0 | 0 | 0 |
| >=20 * ULN | 0 | 0 | 0 | 0 | 0 | 0 |
| Concurrent | | | | | | |
| Elevations | | | | | | |
| ALT or AST | 0 | 0 | 0 | 0 | 0 | 0 |
| >=3 * ULN | | | | | | |
| AND Bilirubin | | | | | | |
| Total >=1.5 * | | | | | | |
| ULN | | | | | | |
| ALT or AST | 0 | 0 | 0 | 0 | 0 | 0 |
| >=3 * ULN | | | | | | |
| AND Bilirubin | | | | | | |
| Total >=2 * | | | | | | |
| ULN | | | | | | |
| Potential Hy's | 0 | 0 | 0 | 0 | 0 | 0 |
| Law | | | | | | |
| ALT or AST | | | | | | |
| >=3 * ULN | | | | | | |
| AND Bilirubin | | | | | | |
| Total >=2 * | | | | | | |
| ULN AND ALP | | | | | | |
| <2 * ULN | | | | | | |

ULN = Upper limit of normal value.
N = number of patients in the Safety Population.
N1 (percentage denominator) = number of patients with at least one non-missing postbaseline value.
Concurrent elevations are from the same day.

TABLE 6
Additional Endpoint: Proportion of Participants with at Least
100% Reduction in Mean Monthly MPM Headache Days by 4-
Week Interval - mITT Population

| | | | Atogepant | Atogepant | | Atogepant | Atogepant |
|---|---|---|---|---|---|---|---|
| | | | 10 mg | 30 mg | Atogepant | 30 mg | 60 mg |
| | | Placebo | QD | QD | 60 mg QD | BID | BID |
| | Statistics | (N = 178) | (N = 92) | (N = 182) | (N = 177) | (N = 79) | (N = 87) |
| Weeks | Responders, | 6 (3.4) | 17 (18.5) | 36 (19.8) | 26 (14.8) | 13 (16.5) | 18 (20.7) |
| 1-4 | n(%) | | | | | | |
| | Non- | 171 (96.6) | 75 (81.5) | 146 (80.2) | 150 (85.2) | 66 (83.5) | 69 (79.3) |
| | responders, n | | | | | | |
| | (%) | | | | | | |
| | Odds ratio | | 6.39 | 7.05 | 4.94 | 5.23 | 7.43 |
| | vs. Placebo | | | | | | |
| | 95% CI | | 2.35, 17.37 | 2.81, 17.68 | 1.93, 12.68 | 1.84, 14.82 | 2.75, 20.11 |
| | Unadjusted | | 0.0003 | <.0001 | 0.0009 | 0.0019 | <.0001 |
| | p-value | | | | | | |
| Weeks | Responders, | 16 (9.7) | 18 (20.9) | 44 (26.7) | 34 (20.1) | 14 (19.7) | 21 (26.3) |
| 5-8 | n (%) | | | | | | |
| | Non- | 149 (90.3) | 68 (79.1) | 121 (73.3) | 135 (79.9) | 57 (80.3) | 59 (73.8) |
| | responders, n | | | | | | |
| | (%) | | | | | | |
| | Odds ratio | | 2.34 | 3.26 | 2.31 | 2.26 | 3.25 |
| | vs. Placebo | | | | | | |
| | 95% CI | | 1.13, 4.87 | 1.76, 6.05 | 1.22, 4.35 | 1.05, 4.89 | 1.59, 6.62 |
| | Unadjusted | | 0.0226 | 0.0002 | 0.0099 | 0.0375 | 0.0012 |
| | p-value | | | | | | |
| Weeks | Responders, | 23 (14.9) | 17 (20.02) | 35 (23.0) | 44 (26.8) | 20 (29.0) | 23 (32.4) |
| 9-12 | n (%) | | | | | | |
| | Non- | 131 (85.1) | 67 (79.8) | 117 (77.0) | 120 (73.2) | 49 (71.0) | 48 (67.6) |
| | responders, n | | | | | | |
| | (%) | | | | | | |
| | Odds ratio | | 1.43 | 1.72 | 2.07 | 2.42 | 2.59 |
| | vs. Placebo | | | | | | |
| | 95% CI | | 0.71, 2.87 | 0.96, 3.07 | 1.18, 3.64 | 1.22, 4.78 | 1.33, 5.04 |
| | Unadjusted | | 0.3110 | 0.0671 | 0.0115 | 0.0111 | 0.0052 |
| | p-value | | | | | | |

Non-responders include participants not met responder criteria.
n = Number of participants within a specific category.
Analyses are based on generalized linear mixed model for repeated measures. The model includes fixed factors (treatment group, analysis visit), covariates (baseline value), and interactions (treatment group by analysis visit, baseline value by analysis visit), with an unstructured covariance matrix (compound symmetry covariance matrix if convergence fails).

What is claimed is:

1. A method of prophylactically treating migraine, comprising the step of administering a prophylactically effective amount an antagonist of Calcitonin Gene-Related Peptide (CGRP-antagonist) selected from atogepant or ubrogepant or a pharmaceutically acceptable salt, ester or prodrug thereof, to a patient in need thereof;

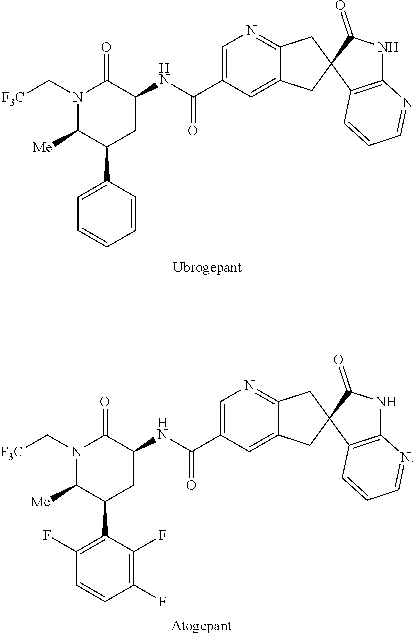 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F ; C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

2. The method according to claim 1 wherein said prophylactic treatment results in at least 50%, 60%, 70%, 75%, 80%, 90%, 98% elimination of migraine.

3. The method according to claim 1 wherein said prophylactic treatment does not result in significant elevation of liver enzymes.

4. The method according to claim 2 wherein said liver enzyme is alanine aminotransferase (ALT) or aspartate aminotransferase (AST) and wherein baseline pretreatment level of AST or ALT is not increased more than 50%, 75%, 100% or 200% after treatment with atogepant or ubrogepant for a period of three months.

5. The method according to claim 3 wherein said liver enzyme is AST and pretreatment baseline level of AST is about 5 to 40 units per liter of serum.

6. The method according to claim 3 wherein said AST levels after treatment with atogepant or ubrogepant, or a pharmaceutically acceptable salt, ester or prodrug thereof, is less than about 100 or 90 or 75 or 60 or 50 units per liter of serum.

7. The method according to claim 3 wherein said liver enzyme is ALT and pretreatment baseline level of AST is about 7 to 56 units per liter of serum.

8. The method according to claim 3 wherein said ALT levels after treatment with atogepant or ubrogepant, or a pharmaceutically acceptable salt, ester or prodrug thereof, is less than about 100 or 90 or 75 or 60 or 50 units per liter of serum.

9. A method of treating migraine in a patient suffering from nonalcoholic steatohepatitis (NASH), comprising the step of administering atogepant or ubrogepant or a pharmaceutically acceptable salt, ester or prodrug thereof, to said patient;

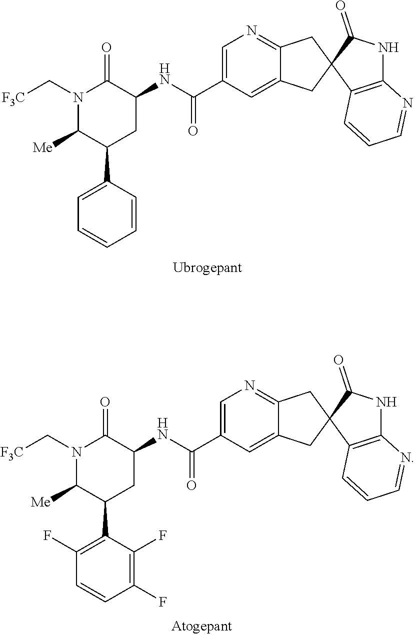 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F ; C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

10. A method of treating migraine in a patient susceptible to treatment with ubrogepant or atogepant comprising the step of administering atogepant or ubrogepant or a pharmaceutically acceptable salt, ester or prodrug thereof, to said patient; 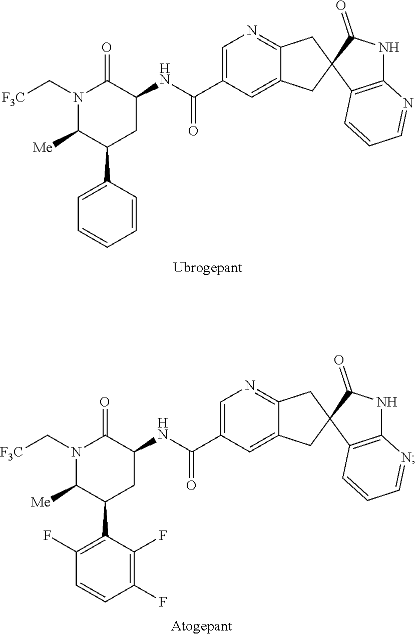 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F ; C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]
Wherein said patient is susceptible to treatment if said patient achieves at least 70% reduction in migraine or probable migraine days after treatment with atogepant or ubrogepant or a pharmaceutically acceptable salt thereof for a period of about three months.

11. The method according to claim 10 wherein said patient achieves at least 75% reduction in migraine or probable migraine days after treatment with atogepant or ubrogepant or a pharmaceutically acceptable salt thereof for a period of about three months.

12. The method according to claim 10 wherein said patient achieves at least 80% reduction in migraine or probable migraine days after treatment with atogepant or ubrogepant or a pharmaceutically acceptable salt thereof for a period of about three months.

13. The method according to claim 10 wherein said patient achieves at least 90% reduction in migraine or probable migraine days after treatment with atogepant or ubrogepant or a pharmaceutically acceptable salt thereof for a period of about three months.

14. The method according to claim 10 wherein said patient achieves 100% reduction in migraine or probable migraine days after treatment with atogepant or ubrogepant or a pharmaceutically acceptable salt thereof for a period of about three months.

15. The method according to claim 4 wherein said treatment is prophylactic.

16. The method according to claim 1 wherein said prophylactic treatment results in a reduction in migraine days per month of at least 0.8, 0.9, 1.0, 1.1, 1.2, 1.3, 1.4, 1.5, 1.6, 1.7, 1.8, 1.9 or 2.0 days per month.

17. The method according to claim 1 wherein said patient experiences reduced frequency or reduced severity of migraine after treatment with ubrogepant or atogepant.

18. A method for the prophylactic treatment of migraine or cluster headaches, the method comprising administering to a patient in need thereof, a prophylactically effective amount of ubrogepant or atogepant, or a pharmaceutically acceptable salt thereof.

19. The method according to claim 1 wherein said patient suffers from one or more symptoms of migraine selected from sinusitis, nausea, nasopharangytis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light, sounds, or smells, worsening pain during physical activity, and vomiting, abdominal pain or heartburn, loss of appetite, lightheadedness, blurred vision, and fainting.

20. The method according to claim 19 wherein said one or more symptoms of migraine is reduced after treatment with ubrogepant or atogepant.

21. The method according to claim 1 wherein atogepant is administered at a dose of about 1-1000 mg per day.

22. The method according to claim 1 wherein atogepant is administered at a dose of about 5, 10, 15, 20, 25, 30, 40, 50, 60, 80, 100, 200, 250, 300, 400 or 500 mg per day.

23. The method according to claim 1 wherein atogepant is administered orally at a once daily dose of about 10 mg.

24. The method according to claim 1 wherein atogepant is administered orally at a once daily dose of about 30 mg.

25. The method according to claim 1 wherein atogepant is administered orally at a once daily dose of about 50 mg.

26. The method according to claim 1 wherein atogepant is administered orally at a once daily dose of about 60 mg.

27. The method according to claim 1 wherein atogepant is administered orally at a once daily dose of about 100 mg.

28. The method according to claim 1 wherein atogepant is administered orally at a dose of about 10 mg two times a day.

29. The method according to claim 1 wherein atogepant is administered orally at a dose of about 30 mg two times a day.

30. The method according to claim 1 wherein atogepant is administered orally at a dose of about 50 mg two times a day.

31. The method according to claim 1 wherein atogepant is administered orally at a dose of about 60 mg two times a day.

32. The method according to claim 1 wherein atogepant is administered orally at a dose of about 100 mg two times a day.

33. The method according to claim 1 wherein ubrogepant is administered at a dose of about 1-1000 mg per day.

34. The method according to claim 1 wherein ubrogepant is administered at a dose of about 5, 10, 25, 50, 100, 200 or 400 mg per day.

35. The method according to claim 1 wherein said CGRP antagonist is atogepant, or a pharmaceutically acceptable, salt, ester or prodrug thereof.
